(19) United States
(12) Patent Application Publication
(10) Pub. No.: US 2023/0381404 A1
(43) Pub. Date: Nov. 30, 2023

(54) USER AUTHENTICATION FOR SETTING AT LEAST ONE INFUSION PUMP

(71) Applicant: B. Braun Melsungen AG, Melsungen (DE)

(72) Inventor: Horst Schmoll, Guxhagen (DE)

(21) Appl. No.: 18/323,067

(22) Filed: May 24, 2023

(30) Foreign Application Priority Data
May 25, 2022 (DE) 10 2022 113 210.2

(51) Int. Cl. A61M 5/142

(52) U.S. Cl. CPC A61M 5/142; A61M 2005/14208; A61M 2205/3553; A61M 2205/502; A61M 2205/6009; A61M 2205/609; A61M 2205/6072

(57) ABSTRACT

A system and method for granting permission clearance to at least one first medical device of a device complex to adjust settings on the first medical device of a device complex. The system has an authentication server unit configured to perform authentication, the first medical device, and an authentication apparatus. The authentication apparatus and/or the first medical device is configured to perform authentication of at least one user. The first medical device and/or the authentication apparatus is configured to communicate with the authentication server unit to perform authentication of the first medical device. Upon successful authentication of the first medical device, the authentication server unit is configured to enable the first medical device with a user-specific use level based on user data.

### CROSS-REFERENCE TO RELATED APPLICATION(S)

This application claims priority under 35 U.S.C. § 119 to German Application No. 10 2022 113 210.2, filed on May 25, 2022, the content of which is incorporated by reference herein in its entirety.

### FIELD

The present disclosure relates to a system and method for granting permission clearance to a medical device or to at least one first medical device of a device complex in order to adjust settings on the one medical device or on the at least one first medical device of a device complex.

### BACKGROUND

Authentication is the general term for checking the authenticity of a proof of identity. In the example of an operating system of a device, which can grant access to a secured area, for example a setting of the device, the user first claims his access permission by entering a previously set, anonymous user name. In addition, the user authenticates himself by entering a password or numeric code, which has also been previously set. The operating system then identifies the user based on this information and subsequently performs authentication, i.e., verification of the provided claim about authenticity. Only when this verification is successful, is the user assigned the defined access authorizations, usually for the duration of a session, as part of the authorization process.

With known user authentication procedures for the use of any device, any person can theoretically access the device in question as long as they have the password/number code and user name. Against this background, however, in the case of a medical device, for example, the actual identity of the user would not be known and could therefore not be recorded for subsequent therapy documentation. With regard to the necessary access restrictions of current medical devices, however, a multi-digit, in particular four-digit numerical code in accordance with generally known user authentication procedures does not satisfy the current security requirements in medical technology. Any person in possession of the corresponding numerical code/password and, if applicable, the anonymous user name would be able to access the medical device, which must not be possible. Rather, it is of considerable importance that only very specific, designated persons can, for example, adjust settings on the respective medical device. For this purpose, such authentication is necessary in order to grant only a specific or selected user(s) permission to adjust various settings on the medical device.

EP 3 087 771 B1 discloses systems, devices, and methods that provide authentication for the operation/use of devices within analyte monitoring systems. The analyte monitoring systems may be in vivo systems and may include a sensor control device having a sensor and accompanying circuitry, and a reader device for communicating with the sensor control device. The analyte monitoring systems may be connected to a trusted computer system located at a remote site.

EP 3 859 573 A1 discloses a method for automatically unlocking and/or locking a computer-based medical product in a system. The system comprises the computer-based medical product and a mobile terminal device, wherein the mobile terminal device comprises a first wireless communication device for wireless transmission and reception of data, wherein mobile application software is executable via the mobile terminal device. The medical product comprises a device computer, a second wireless communication device for wireless transmission and reception of data, wherein a driver software is executable on the device computer, which can establish a wireless communication connection to the first communication device of the mobile terminal device via the second communication device of the medical product. In this regard, it is provided that the medical product is automatically switched from a locked state to an unlocked state when the first communication device of the mobile terminal device has established a wireless connection with the second communication device of the medical product and the signal strength of the signal of the first communication device of the mobile terminal device received by the second communication device of the medical product exceeds a predetermined first threshold value and/or the medical product is automatically switched from an unlocked state to a locked state, when the first communication device of the mobile terminal device has established a wireless connection with the second communication device of the medical product and the signal strength of the signal of the first communication device of the mobile terminal device received by the communication device of the medical product falls below a predetermined second threshold value and/or if the wireless connection is interrupted for longer than a predetermined first time interval.

Finally, US 2017 0 140 134 describes an exemplary medical device. The device includes a physiological measurement device, a device management engine, a user caching engine, and a login engine. The device management engine is configured to receive data acquired from the physiological measurement device. The user caching engine is configured to store cache records linked with users in a user cache. The login engine is configured to receive a user ID that is hardcoded to a particular user and to determine whether the user ID is associated with a cache record stored in the user cache. If it is determined that the user ID is linked with a cache record stored in the user cache, the login engine is configured to log the user in. If it is not determined that the user ID is linked with an unexpired cache record that is stored in the user cache, the login engine is configured to prompt the user for proof of authorization.

### SUMMARY

The present disclosure is based on the object of providing a system that enables simple, fast and user-dependent, secure authentication. Furthermore, it is in particular a goal of the present disclosure to eliminate or at least improve disadvantages of the prior art.

Accordingly, the system for granting permission clearance to one medical device or to at least one first medical device of a device complex, in particular an infusion pump, in order to adjust settings on the one medical device or on the at least one first medical device of a device complex, has an authentication server unit provided and configured to perform authentication, the one medical device or the at least one first medical device, and an authentication apparatus, preferably in the form of a mobile terminal device/a mobile terminal device. Here, the authentication apparatus and/or the one medical device or the at least one first medical device is provided and configured to perform authentication of at least one user, wherein the one medical device or the at least one first medical device and/or the authentication apparatus is provided and configured to communicate with the authentication server unit to perform authentication of the one medical device or the at least one first medical device. Upon successful authentication of the one medical device or of the at least one first medical device, the authentication server unit is provided and configured to enable the one medical device or the at least one first medical device with a user-specific use level based on user data.

In other words, the system grants setting of/on the one medical device or of/on the at least one first medical device, in particular an infusion pump. Here, an authentication apparatus is provided, in which a user enters his login data (login information) or user name and password, respectively. Alternatively or additionally, the login data or the user name and password may also be entered directly at the medical device, if it is a single device, or directly at a first medical device, if it is a device complex. In such a device complex, a first medical device is provided, which is the so-called main device, i.e. the medical device with the help of which the authentication and authenticating is carried out or with which a first communication or a first input takes place. After successful authentication, the login data or the user name and password are forwarded/sent to the authentication server unit in order to perform authentication, i.e. to check whether the user name and password are correct and whether granting a permission clearance should be permitted. If authentication is successful, a permission clearance is granted to the user together with a corresponding user-specific use level, which is preferably stored in the authentication server unit.

In other words, a system for user authentication is provided for one medical device or a group of medical devices. The initial authentication may be performed via an authentication apparatus/mobile application or (directly) on the one medical device or on the one first medical device. After successful authentication with the aid of the authentication server unit, the user level assigned to a user grants the user corresponding access to a user interface of the medical device or of the at least one first medical device, respectively. An authentication apparatus/mobile application communicates with the authentication server unit/with an IT server. The one medical device or the at least one first medical device is also connected to the authentication server unit and bidirectional communication is established. The user enters the user data into the authentication device.

Alternatively, it is also possible for the user data to be entered directly into the first medical device.

In both cases, it is advantageous if username and password or biometric data are used for user authentication. It is preferred if the authentication apparatus is provided and configured so that the data for user authentication can be entered therein. This user data/login data is sent to the authentication server unit either by the authentication apparatus or by the one/first medical device.

It is preferred if the authentication server unit is provided and configured to unlock at least one further medical device in communication contact with the at least one first medical device from the device complex, preferably with the user-specific use level.

In other words, the authentication server unit sends an authentication command to at least one further medical device or respectively to all medical devices in communication contact with the first medical device. That is, it is preferred if the further medical devices or the group of medical devices belongs to the first medical device. Here, a group is 1 to n medical devices, which are identifiable by the server and belong, for example, to the same bed location or to the same patient, or are arranged in an (infusion pump) rack.

It is advantageous if the one medical device or the at least one first medical device or the authentication apparatus is provided and configured to enter the user data therein and send it to the authentication server unit.

It is preferred if the authentication server unit is provided and configured to check and validate the entered data. After successful authentication, the user is linked to his user profile, which contains/has use levels/the use level for the one medical device or the at least one first medical device.

It is advantageous if the authentication server unit is provided and configured to send an authentication command to the at least one further medical device, wherein the authentication command comprises information, preferably at least a user name, a time stamp and the user-specific use level. In other words, the authentication command comprises at least a user name, a user level/use level and a time stamp.

It is preferred if the one medical device or the at least one first medical device is provided and configured to display a code, preferably a QR code or barcode, which is provided and configured to output a device identification of the at least one first medical device by scanning via the authentication apparatus.

It is advantageous if a communication connection between the authentication server unit and the one medical device or the at least one first medical device is configured bidirectionally.

It is advantageous if the at least one further medical device (2) is provided and configured to store the information of the authentication command, preferably in a test protocol of the at least one further medical device. In other words, after receiving the command/authentication command, the at least one further medical device is provided and configured to store this command in the test protocol of the at least one further medical device and to grant access to a user interface according to the received use level/user level.

In other words, it is advantageous that if the user has already logged in to a single or first device, the identification of the one medical device is already known to the authentication server unit. In the case of the mobile application/authentication apparatus, the user enters the identification number of the medical device, for example by manually entering a number or by scanning a barcode containing the number. It is possible to use technologies such as RFID, Bluetooth or NFC for device identification.

It is preferred if the system is provided and configured to enable processing of the one medical device or of the at least one first and/or of the at least one further medical device without authentication in an emergency situation, preferably at a lowest use level.

In other words, in emergency situations, the medical device has to allow access at the lowest use level without authentication. In this case, an anonymous user name is stored in the test protocol, and only a mandatory subset of the device functions associated with the lowest use level is available to the user.

It is advantageous if the authentication device is configured and provided to control at least parts of the functionality of the at least one medical device or of the group of medical devices, respectively.

It is advantageous if the at least one medical device is provided to require re-authentication after a certain period of inactivity.

Furthermore, the present disclosure relates to a method for granting permission clearance to a medical device or to at least one first medical device of a device complex, in particular an infusion pump, in order to adjust settings on the one medical device or on the at least one first medical device of a device complex, comprising the following steps:
entering user data, preferably user name and password or biometric data, into an authentication apparatus and/or the one medical device or into the at least one first medical device;
sending the input to an authentication server unit;
entering a device identification of the one medical device or of the at least first medical device into the authentication apparatus, preferably by scanning a code displayable on the one medical device or on the at least first medical device;
performing the authentication on the one medical device or on the at least one first medical device; and
granting the permission clearance and assigning a user-specific use level.

It is preferred if the method comprises the following steps:
sending an authentication command to at least one further medical device;
storing the received information of the authentication command in the further medical device; and
using the at least one further medical device according to the received use level.

In other words, a user enters their user data into the authentication apparatus/mobile application or into a single (first) medical device. The user data is sent from the authentication apparatus or from the single (first) medical device to the authentication server unit and grants the user access to the corresponding medical device.

The authenticated user is assigned a use rank/use level for the corresponding medical device. Subsequently, the device identification is then entered in the mobile application. The input is preferably made by manual input or by scanning a barcode or via Bluetooth.

The authentication server unit sends an authentication command to all further medical devices in communication with the first medical device or belonging to the device group, respectively. The authentication command contains the user name, the use level and the time stamp.

The medical devices store the received user name and the use level in their test protocol. The medical devices allow the use according to the received user level.

After a certain period of inactivity, the use of the device is blocked again, the information is stored in the test protocol.

### BRIEF DESCRIPTION OF THE DRAWINGS

FIG. 1 is a diagram illustrating a system exemplified by a group of medical devices or infusion pumps, respectively, in accordance with an embodiment of the present disclosure; and

FIG. 2 is a flowchart of the method according to the present disclosure.

### DETAILED DESCRIPTION

Configuration examples of the present disclosure are described below based on the accompanying figures.

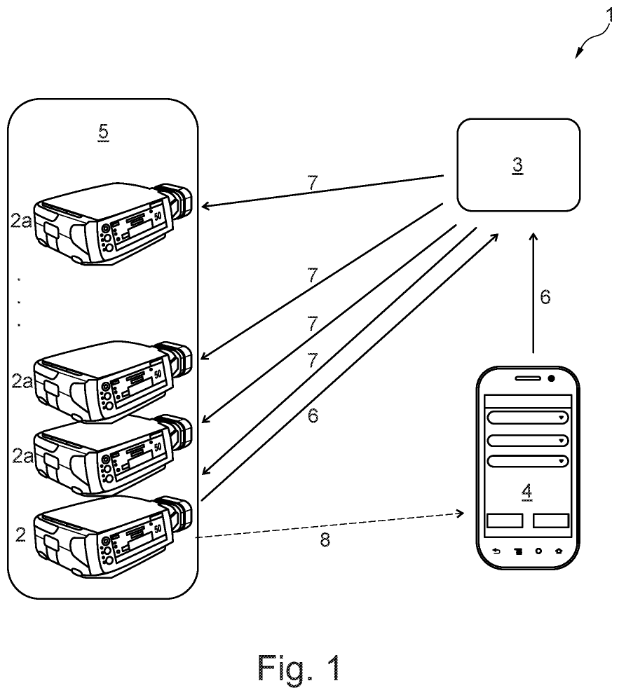

FIG. 1 is a diagram illustrating a system 1 according to an embodiment of the present disclosure. FIG. 1 shows a first medical device 2, an authentication server unit 3, and an authentication apparatus 4. Furthermore, FIG. 1 shows further medical devices 2a which are in communication contact with the first medical device 2.

The first medical device 2 and the other medical devices 2a in communication contact with it are preferably arranged in a device complex/rack 5 or at least in one room and associated with one patient.

The first medical device 2 and/or the authentication apparatus 4 is/are provided and configured to communicate with the authentication server unit 3 for performing authentication of the first medical device 2.

Upon successful authentication of the first medical device 2, the authentication server unit 3 is provided and configured to unlock the first medical device 2 with a user-specific use level based on entered user data.

A first communication connection 6 is provided between the first medical device 2 and the authentication server unit 3. Additionally or alternatively, a first communication connection 6 is provided between the authentication apparatus 4 and the authentication server unit 3. The authentication server unit 3 has a respective second communication connection 7 with the first medical device 2 and the further medical devices 2a.

The first communication connection 6 is configured and provided to send input data entered by a user to the authentication server unit 3. The second communication connection 7 is configured and provided to send an authentication command to the first medical device 2 and the further medical devices 2a. The communication connections 6 and 7, which connect the first medical device 2 and the authentication server unit 3, are therefore a bidirectional communication connection.

As shown in FIG. 1, the authentication apparatus 4 is provided and configured to perform a device identification 8. The device identification 8 is performed via a manual input or scanning of a barcode or via Bluetooth.

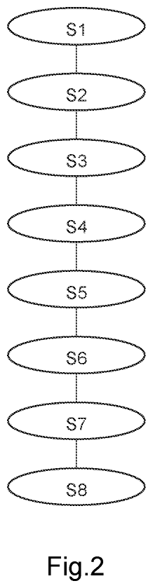

FIG. 2 is a flowchart of the method according to the present disclosure.

In a first step S1, user data, preferably user name and password or biometric data, is input into the authentication apparatus 4 or into at least a first medical device 2.

In a second step S2, the input is sent to an authentication server unit 3. The input received is checked and validated by the authentication server unit 3.

In a third step S3, a device identification 8 of the first medical device 2 is performed. This is done by entering the device identification 8 into the authentication apparatus 4, preferably by scanning a code that can be displayed on the first medical device 2.

In a subsequent step S4, the first medical device 2 is authenticated and in a step S5, a user-specific use level is assigned to the first medical device 2.

In a step S6, the authentication server unit 3 sends an authentication command to at least one further medical device 2a.

In step S7, the received information of the authentication command is stored in a test protocol of each medical device 2 and 2a. The information is at least a user name, the use level and a time stamp.

In a final step S8, the user uses the at least one medical device 2 and/or 2a according to the received use level to adjust a setting. Using the at least one medical device 2 and/or 2a means setting existing and/or required parameters.

1.-10. (canceled)

11. A system for granting permission clearance to at least one first medical device of a device complex to adjust settings on the at least one first medical device, the system comprising:
an authentication server unit for performing authentication;
the at least one first medical device; and
an authentication apparatus,
the authentication apparatus and/or the at least one first medical device being configured to perform authentication of at least one user,
the authentication apparatus and/or the at least one first medical device being further configured to communicate with the authentication server unit to perform authentication of the at least one first medical device,
the authentication server unit being configured to enable the at least one first medical device with a user-specific use level based on user data upon successful authentication of the at least one first medical device.

12. The system according to claim 11, wherein the at least one first medical device is an infusion pump.

13. The system according to claim 11, wherein the authentication server unit is provided and configured to unlock at least one second medical device in communication contact with the at least one first medical device from the device complex.

14. The system according to claim 13, wherein the authentication server is provided and configured to unlock the at least one second medical device with the user-specific use level.

15. The system according to claim 13, wherein the authentication server unit is provided and configured to send an authentication command to the at least one second medical device, wherein the authentication command comprises information.

16. The system according to claim 15, wherein the information comprises at least a user name, a time stamp and the user-specific use level.

17. The system according to claim 15, wherein the at least one second medical device is provided and configured to store the information.

18. The system according to claim 17, wherein the information is stored in a test protocol of the at least one second medical device.

19. The system according to claim 13, wherein the system is provided and configured to enable processing of the at least one first medical device and/or of the at least one second medical device without authentication in an emergency situation.

20. The system according to claim 19, wherein the processing is enabled at a lowest use level.

21. The system according to claim 11, wherein the at least one first medical device or the authentication apparatus is provided and configured to enter user data and send user data to the authentication server unit.

22. The system according to claim 11, wherein the at least one first medical device is provided and configured to display a code, which is provided and configured to output a device identification of the at least one first medical device by scanning via the authentication apparatus.

23. The system according to claim 22, wherein the code is a QR code or a barcode.

24. The system according to claim 11, wherein a communication connection between the authentication server unit and the at least one first medical device is configured bidirectionally.

25. A method for granting a permission clearance to at least one first medical device of a device complex in order to adjust settings on the at least one first medical device of a device complex, the method comprising the steps of:
entering user data into an authentication apparatus and/or the at least one first medical device;
sending the user data to an authentication server unit;
entering a device identification of the at least one first medical device into the authentication apparatus;
performing an authentication on the at least one first medical device; and
granting the permission clearance and assigning a user-specific use level.

26. The method according to claim 25, wherein the at least one first medical device is an infusion pump.

27. The method according to claim 25, wherein the user data is a user name and password or biometric data.

28. The method according to claim 25, wherein the device identification is entered by scanning a code displayable on the at least one first medical device

29. The method according to claim 25, further comprising the steps of:
sending an authentication command to at least one second medical device;
storing received information of the authentication command in the at least one second medical device; and
using the at least one second medical device according to a received use level.
